(19) United States
(12) Patent Application Publication
(10) Pub. No.: US 2002/0171806 A1
(43) Pub. Date: Nov. 21, 2002

(54) Optical measurement device

(76) Inventor: Morton Z. Baumgarten, Middletown, NJ (US)

Correspondence Address: Charles I. Brodsky, Esq., 2 Bucks Lane, Marlboro, NJ, 07746, US

(21) Appl. No.: 09/861,486

(22) Filed: May 21, 2001

(51) Int. Cl. A61B 3/04

(52) U.S. Cl. 351/227

(57) ABSTRACT

An optical measurement device including a frame to be supported by the nose and ears of a wearer, right and left lenses set within the frame, a pair of adjustable right and left lenses set within quiescent position a fixed distance above a bottom edge of the lenses measured vertically, and an orthogonal grid on a surface of each of the lenses extending both vertically and horizontally, all allowing X and Y coordinate information of pupil location to be determined according to wearer comfort position, in facilitating the on-line ordering of eyewear products.

### FIELD OF THE INVENTION

This invention relates to such eyewear products as eyeglasses, sunglasses and sports vision equipment for both prescription and non-prescription purposes, in general, and to an optical measurement device useful in the ordering of such eyewear products, in particular.

### BACKGROUND OF THE INVENTION

As is well known and understood, more and more consumer products are everyday becoming more available for purchase online, through the Internet. Suggestions have been proposed for extending this to the ordering of eyewear products interactively. According to such proposals, an image of a potential customer is scanned onto a computer screen, and images of available frames are then captured and overlaid onto the facial image to present a virtual picture of what the customer might look like when wearing each selected frame. However, and as will be appreciated, the potential customer would still have to visit an eye doctor to obtain a current prescription, where one is needed. Moreover, even if the eyewear product is of a type for which no prescription is required, the prospective customer still visits the eye doctor—or such large eyewear establishments as Pearle Vision, Lens Crafters and Cohen Optical—so as to be measured for the frames to be ordered. Whether the fitting be for such prescription eyewear products as single vision glasses or bifocal glasses (inclusive of progressive bifocals)—or whether they be merely of non-prescription sports vision equipment—, a second and/or subsequent visit needs to be had if a second, third, or additional pair of glasses is to be later purchased. One thought behind these proposals of ordering eyewear products on-line is the system's storage of data characterizing the prospective customer to avoid these subsequent visits, to the extent that later purchases can be made interactively from the home or from kiosks in a mall, as well as from the eye doctor's office.

As will be apparent, some manner of taking these measurements must still be had for fitting any frame selected to the face of the eyewear product wearer. Having such measurements taken at the eye doctor's office or at a Vision Center involves the participation of a trained professional. With the teachings of the present invention, on the other hand, even laypersons become competent to take the needed measurements, in allowing inputs to a database to be made from the home or from kiosk units placed at various strategic points of purchase. With the teachings of the invention, moreover, such measurements for proper fitting can continue to be made at the eye doctor's office or at the Vision Center, but can there be taken by a receptionist even after only a few minutes of training. In any event, once the measurements are taken, they are fed into the system database, so as to be accessible at a later time in ordering a subsequent pair of glasses, without the need for any re-visit to the eye doctor, as long as any prescription information is up-to-date (if such prescription information is, indeed, needed).

As will be appreciated by those skilled in the art, with the teachings of the present invention, the end result becomes both a proper scaling of the virtual eyewear down-loaded onto the image displayed on the computer screen of the customer, and all the information needed by a manufacturer to properly fit the frame to the face; and, at least as important, to fit the frame in the way the customer is most comfortable wearing it (i.e., where along the nose the glasses are to sit).

As will be seen, the teachings of the invention thus allow customers to try on glasses via the Internet. Using the invention, individuals will be able to create images of themselves that they can view while on-line—enabling them to “try on” and select any eyewear product from the virtual eyewear inventory of a manufacturer. Once a frame has been selected, prescription lenses can then be manufactured into the glasses, where appropriate—and mailed directly to the purchasers' homes. And, as will be seen, this will be understood to be available both to purchasers of single vision glasses, bifocal glasses, progressive bifocal glasses, sunglasses, sports vision glasses, etc., for both prescription and non-prescription purchases.

### SUMMARY OF THE INVENTION

As will become clear from the following description, the teachings of the invention follow from a recognition that the two most important measurements to be made in the fitting of glasses are the distance between the pupils of the wearer (the “pupillary distance”) and the distance between the center line of the pupil and the bottom edge of the lens (especially where a bifocal pair of glasses is involved).

As the manufacturer of the eyewear frame typically sets the distance between the nose pads and the bottom edge of the frame, and the distance between the nose pads and the adjacent edge of the frame, all that becomes necessary for a proper fitting is to determine the location of the center of the pupil with respect to the nose pad once an eyeglass measurement device is placed on the wearer, and fitted along the nose at the position where the wearer feels most comfortable with the device in place. Rather than having the eye doctor or optician look through each lens and then measure these distances for forwarding to the eyeglass manufacturer, the present invention will be seen to effectuate this with a grid in the form of a recognition module able to determine measurements within tolerances mandated in the optical industry. Readings of the grid can then be obtained even by a layperson with a minimum amount of training, for forwarding to the eyeglass manufacturer interactively, along with the name, address, telephone number, social security number, etc. of the customer ordering the eyewear.

Thus, and as will be understood from the following description, the present invention is in the form of an optical measuring device having a frame to be supported about the nose and eyes of a wearer, right and left lenses set within the frame, a pair of adjustable right and left nose pads in quiescent position a fixed distance above a bottom edge of the lenses measured vertically, and a orthogonal grid on a surface of each of the lenses extending both vertically and horizontally. With the right and left nose pads adjustable to align substantially parallel to one another in the quiescent position, the optical measurement device of the invention further includes a scissor adjust hinged with the right and left nose pads for angularly spreading the nose pads from the quiescent position in accordance with the location on the wearer's nose where the frame is desired to sit and most comfortably worn. The optical measuring device also includes a pair of springs coupled between the frame and scissor adjust for limiting the angular spread of the right and left nose pads—both for fitting the frame on the nose and for retaining the right and left nose pads in the substantially parallel, quiescent position under the initial bias provided to the spring. In a preferred embodiment of the invention to be described, each of the orthogonal grids includes a plurality of rows and columns, respectively spaced a predetermined distance apart from one another within the range 0.5 to 1.0 mm—well within the 0.8 mm range mandated for quality assurance in the optical industry.

In the preferred embodiment of the optical measurement device to be described, one of the columns of each orthogonal grid is positioned a fixed distance from a center line between the right and left nose pads when in their substantially parallel, quiescent position. At the same time, in this embodiment, one of the rows of each orthogonal grid is also positioned co-linearly with a center line through the right and left nose pads—again, when in their substantially parallel position. As will be understood, such constraints allow for calculation of the pupillary distance of the wearer as well as the distance from the center of the pupil to the bottom edge of the lens as a critical calculation in bifocal design, independent of where the wearer feels most comfortable in setting the frame along the length of the nose. With each column and each row of the orthogonal grid being assigned an identifying coordinate, all that becomes necessary for correct fitting to be had is to read off the X and Y coordinates where the center of the pupil falls behind the grid with the frames being worn.

### BRIEF DESCRIPTION OF THE DRAWINGS

These and other features of the invention will be more clearly understood from a consideration of the following description, taken in connection with the accompanying drawings, in which:

FIG. 1 is a front perspective view of a preferred embodiment of the optical measurement device of the invention helpful in an understanding of its features; and

FIG. 2 is side perspective view of the optical measurement device of FIG. 1.

### DETAILED DESCRIPTION OF THE INVENTION

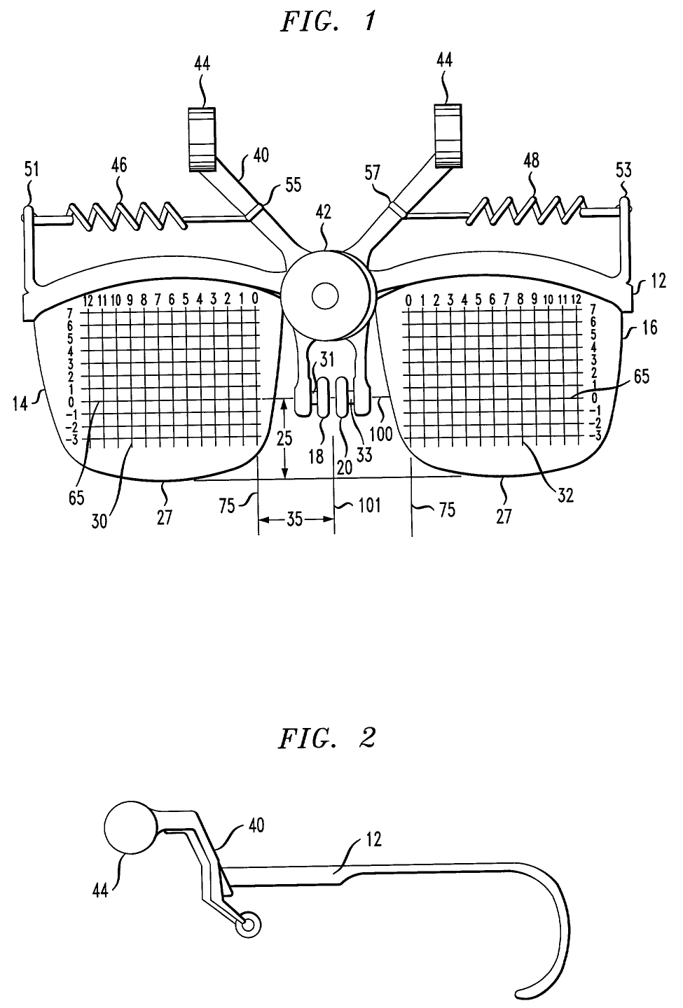

Referring to the drawings, an optical measurement device according to the invention includes a frame 12 to be supported about the nose and ears of a wearer, along with right and left lenses 14, 16 set within the frame 12 in any appropriate manner. A pair of adjustable right and left nose pads 18, 20 are included, in quiescent position a fixed distance 25 above a bottom edge 27 of the lenses 14, 16 measured vertically. An orthogonal grid is included on a surface of each of the lenses 14, 16 extending both vertically and horizontally, shown in FIG. 1 as 30, 32, respectively. As illustrated, the right and left nose pads 18, 20 are adjustable about pivot points 31, 33, and align substantially parallel to one another in the quiescent position shown in FIG. 1.

In a preferred embodiment of the invention, a scissor adjust 40 couples with the right and left nose pads 18, 20 by a hinge 42 so as to allow angular spreading of the nose pads 18, 20 by pinching end points 44 of the scissor adjust toward one another. A pair of springs 46, 48 are included, coupled between the frame at 51, 53 and the scissor adjust 40 at 55, 57 for limiting the angular spread of the right and left nose pads 18, 20 to an extent consistent with the breadth of nose sizes typically encountered. As indicated in FIG. 1, the springs 46, 48 are initially biased to retain the right and left nose pads 18, 20 in the substantially parallel, quiescent condition shown.

As will become clear from the following description, the teachings of the invention follow from the use of orthogonal grids 30, 32 being composed of a plurality of rows and columns, each respectively spaced a predetermined distance apart. Thus, as an example, each respective row of the grids 30, 32 may be spaced apart a distance within the range 0.5 to 1.0 mm, while each respective column is similarly spaced apart a like distance. As indicated, one such row 65 on the lenses 14, 16 is positioned co-linearly with a center line 100 through the right and left nose pads 18, 20 in the substantially parallel, quiescent position. At the same time, one of the columns 75 in the two grids is positioned a fixed distance 35 from a center line 101 between the nose pads 18, 20 when in the quiescent position.

To use the optical measurement device of FIGS. 1 and 2, all that needs to be done is for the wearer to put on the frame 12 and to set the scissor adjust 40 to that position on the nose where the frame feels comfortable, and where the wearer typically would position glasses, sunglasses, sports vision equipment, etc. to be worn. Once so situated, the wearer need only look in a mirror to see where the center of his/her pupil aligns within the grid 30, 32, and note the X and Y coordinates for each grid (something which can be done at home, or at a kiosk unit at a mall). Obviously, the same could also be done in an eye doctor's office, either by the prospective customer alone, or with the assistance of clerical personnel. Then, with the distance 35 from column 75 to the center line 101 known, and with the spacing between each column of the grids 30, 32 known, the distance between the wearer's pupils at the comfort position could easily be calculated for the manufacture of single vision or bifocal lenses for the particular design frame selected. That is, with the distance from the center line 100 through the nose pads 18, 20 to the bottom edge 27 of the lenses 14, 16 known (as provided by the manufacturer of the frame for each frame chosen), and with the spacing between each row of the grids 30, 32 known, the distance between the center of the pupil and the bottom edge 27 could be calculated for the frame selected simply by noting on which row of the grids 30, 32 the center of the pupil aligns, for that comfort wearing position.

As will be appreciated by those skilled in the art, these calculations can be had through a simple computer program at the manufacturer's location to which the coordinate information is interactively transmitted and thereafter stored. With a further identification of the wearer known, the manufacturer would then be in a simple position to forward the manufactured eyewear along, by any appropriate manner. Of course, prescription information would need be required as well for prescription eyewear—but as long as previously provided prescription information is current, the X-Y coordinate information could be sent on-line by a customer at any time, for ordering a second, or different pair of eyewear—once again, in accordance with the frame selected, and in accordance with the comfort position of the nose pads—either forwardly toward the tip of the nose, or rearwardly toward the bridge of the nose, and in accordance with the breadth and size of the wearer's nose.

While there has been described what is considered to be a preferred embodiment of the present invention, it will be readily appreciated by those skilled in the art that modifications may be made without departing from the scope of the teachings herein. For example, while the preferred embodiment has proceeded with the use of orthogonal grids in conjunction with a frame measurement device in which the right and left nose pads 18, 20 are quiescently placed in substantially parallel position, it will be apparent that a quiescent position can be other than “substantially parallel”, or that the nose pads be “spring-loaded”. For at least such reason, resort should be had to the claims appended hereto for a true understanding of the scope of the invention, which serves to allow customers to try on and purchase both frames and lenses over the Internet. Whether the digital image of the customer is captured at home, at a kiosk, at an eye doctor's office, or at a Vision Center, it is then stored in the manufacturer's system database, according to the invention, to allow accessibility from all locations in allowing the customer to try on complete catalogues of eyewear products and to place orders interactively.

I claim:

1. An optical measurement device comprising a frame to be supported by the nose and ears of a wearer, right and left lenses set within said frame, a pair of adjustable right and left nose pads in quiescent position a fixed distance above a bottom edge of said lenses measured vertically, and an orthogonal grid on a surface of each of said lenses extending both vertically and horizontally.

2. The optical measurement device of claim 1 wherein said right and left nose pads are adjustable to align substantially parallel to one another in said quiescent position.

3. The optical measurement device of claim 2, including a scissor adjust hinged with said right and left nose pads for angularly spreading said nose pads from said quiescent position.

4. The optical measurement device of claim 3, also including a pair of springs coupled between said frame and said scissor adjust for limiting angular spread of said right and left nose pads.

5. The optical measurement device of claim 4 wherein said pair of springs are initially biased to retain said right and left nose pads in said substantially parallel, quiescent position.

6. The optical measurement device of claim 1 wherein each of said orthogonal grids includes a plurality of rows and columns respectively spaced a predetermined distance apart.

7. The optical measurement device of claim 6 wherein each respective row is spaced apart a distance within the range 0.5 to 1.0 mm.

8. The optical measurement device of claim 6 wherein each respective column is spaced apart a distance within the range 0.5 to 1.0 mm.

9. The optical measurement device of claim 6 wherein one of said columns of each orthogonal grid is positioned a fixed distance from a center line between said right and left nose pads in said substantially parallel, quiescent position.

10. The optical measurement device of claim 6 wherein one of said rows of each orthogonal grid is positioned co-linearly with a center line through said right and left nose pads in said substantially parallel, quiescent position.

11. The optical measurement device of claim 5 wherein each of said orthogonal grids includes a plurality of rows and columns respectively spaced a predetermined distance apart.

12. The optical measurement device of claim 11 wherein each respective row is spaced apart a distance within the range 0.5 to 1.0 mm.

13. The optical measurement device of claim 12 wherein each respective column is spaced apart a distance within the range 0.5 to 1.0 mm.

14. The optical measurement device of claim 13 wherein one of said columns of each orthogonal grid is positioned a fixed distance from a center line between said right and left nose pads in said substantially parallel, quiescent position.

15. The optical measurement device of claim 14 wherein one of said rows of each orthogonal grid is positioned co-linearly with a center line through said right and left nose pads in said substantially parallel, quiescent position.

16. The optical measurement device of claim 9 wherein said position of, and said spacing between, said columns are selected for providing an indication of distance between pupils of the eyes of said wearer.

17. The optical measurement device of claim 14 wherein said position of, and said spacing between, said columns are selected for providing an indication of distance between pupils of the eyes of said wearer.

18. The optical measurement device of claim 10 wherein said position of, and said spacing between, said rows are selected for providing an indication of distance between said bottom edges of said lenses and pupils of the eyes of said wearer.

19. The optical measurement device of claim 15 wherein said position of, and said spacing between, said rows are selected for providing an indication of distance between said center line through said right and left nose pads and the pupils of the eyes of said wearer.
